(19) United States
(12) Patent Application Publication
(10) Pub. No.: US 2022/0185076 A1
(43) Pub. Date: Jun. 16, 2022

(54) NEBULIZER SYSTEM FOR A MOTOR VEHICLE

(71) Applicant: Valeo Systemes Thermiques, Le Mesnil Saint-Denis (FR)

(72) Inventors: Georges De Pelsemaeker, Le Mesnil Saint-Denis (FR); Yves Rousseau, Le Mesnil Saint-Denis (FR); Vincent Feuillard, Le Mesnil Saint-Denis (FR); Antoine Roddes, Le Mesnil Saint-Denis (FR); Mohamed-Amine Boubaker, Le Mesnil Saint-Denis (FR)

(73) Assignee: Valeo Systemes Thermiques, Le Mesnil Saint-Denis (FR)

(21) Appl. No.: 17/605,359

(22) PCT Filed: Feb. 11, 2020
(86) PCT No.: PCT/FR2020/050244

§ 371 Date: Oct. 21, 2021

(30) Foreign Application Priority Data
Apr. 24, 2019 (FR) 1904294

(51) Int. Cl. B60H 3/02

(52) U.S. Cl. CPC B60H 3/022; B60H 2001/006

(57) ABSTRACT

The invention relates to a nebulizer system (1) for a motor vehicle. It comprises: a. at least one reservoir (10) for a liquid; b. at least one nebulization chamber (14) equipped with a nebulizer nozzle (3), said nozzle being equipped with a device (4) for emitting acoustic waves, for example a piezo-electric element (4), said nebulization chamber (14) being configured in such a way that the liquid coming from the reservoir (10) forms a jet (13) of liquid and a mist (12) of droplets, said mist being intended to enter a motor vehicle interior (2); c. at least one acoustic attenuation means (20). According to the invention, the acoustic attenuation means (20) comprises at least one grating (20).

The present invention relates to a nebulizer system for a motor vehicle. This system is intended more particularly for the generation of fine droplets for cooling and/or humidifying a vector flow, in particular an air flow, so as to form a cooling mist. The invention also relates to a ventilation, heating and/or air-conditioning device for a motor vehicle equipped with such a nebulizer system.

In a motor vehicle, even one provided with a ventilation and/or air-conditioning device, it is not uncommon for the users of the vehicle to suffer under heat in the vehicle interior, in particular the rear seat passengers, who are farther away from the air vents of the ventilation and/or air-conditioning device.

As a result, it is advantageous to make use of a nebulizer system, since the mist of water rapidly cools the air in the vehicle interior, providing a sensation of immediate cold.

During operation, the existing nebulizer systems generate noise and vibrations that can have a negative effect on the comfort of the users of the vehicle. In particular, the liquid jet, for example the water jet generated by the system, brings about flow noise when it strikes one of the walls of the system.

The present invention aims to improve the situation by proposing an optimized nebulizer system.

To this end, the subject of the invention is a nebulizer system for a motor vehicle, said system having:
a. at least one reservoir for a liquid,
b. at least one nebulizer enclosure provided with a nebulizer nozzle, said nozzle being provided with a device for emitting acoustic waves, for example a piezoelectric element, said nebulizer enclosure being configured such that the liquid coming from the reservoir forms a liquid jet and a mist of droplets, said mist being intended to pass into a motor vehicle interior,
c. at least one acoustic attenuation means,

said system being characterized in that said acoustic attenuation means comprises at least one grating.

By virtue of such an acoustic attenuation means, it is possible to significantly reduce the noise generated by the liquid jet when it strikes one of the walls of the nebulizer system.

According to various features of the invention which may be considered together or separately:
the nebulizer nozzle forms a concentrator for the acoustic waves generated by the piezoelectric element;
the nebulizer enclosure comprises a nebulizer chamber and a nebulizer tube, the nebulizer nozzle being inserted at least partially into said nebulizer chamber and the grating being disposed at least partially in the nebulizer tube;
the grating conforms to the shape of at least one of the walls of the nebulizer tube;
the wall is a bottom wall of the nebulizer tube;
the bottom wall of the nebulizer tube comprises a channel intended to receive the liquid jet output by the nebulizer nozzle;
the grating is disposed above the channel;
the grating at least partially conforms to the shape of the channel;
the grating extends in a plane that is inclined with respect to the bottom wall of the nebulizer tube;
the nebulizer tube comprises at least one guide means protruding from one from one of the walls of the nebulizer tube, said guide means being intended to guide the grating along the inclined plane;
the guide means is a slideway;
the guide means comprises a set of bearing points arranged on one of the walls of the nebulizer tube;
the guide means is integral with the wall of the nebulizer tube;
the gratings are arranged in pairs and the gratings of one and the same pair are in contact with one another;
the gratings are turned at an angle of 45° with respect to one another;
the gratings are arranged at a distance from one another;
the grating is made up of a single part or of a plurality of parts placed end to end;
the nebulizer tube has at least one outlet duct for the mist;
the nebulizer tube has a single outlet duct for the mist;
the grating is made of flexible or rigid plastics material;
the grating is made of metal or metal alloy, for example stainless steel;
the grating is made of hydrophobic material;
the grating is produced from woven threads;
the grating is produced from a perforated metal sheet;
the grating has a mesh of circular, triangular, rectangular, square or hexagonal type.

Another subject of the invention is a ventilation, heating and/or air-conditioning device for a motor vehicle, comprising a nebulizer system as described above.

Further features and advantages of the invention will become apparent on reading the following description. This description is purely illustrative and should be read in conjunction with the appended drawings, in which:

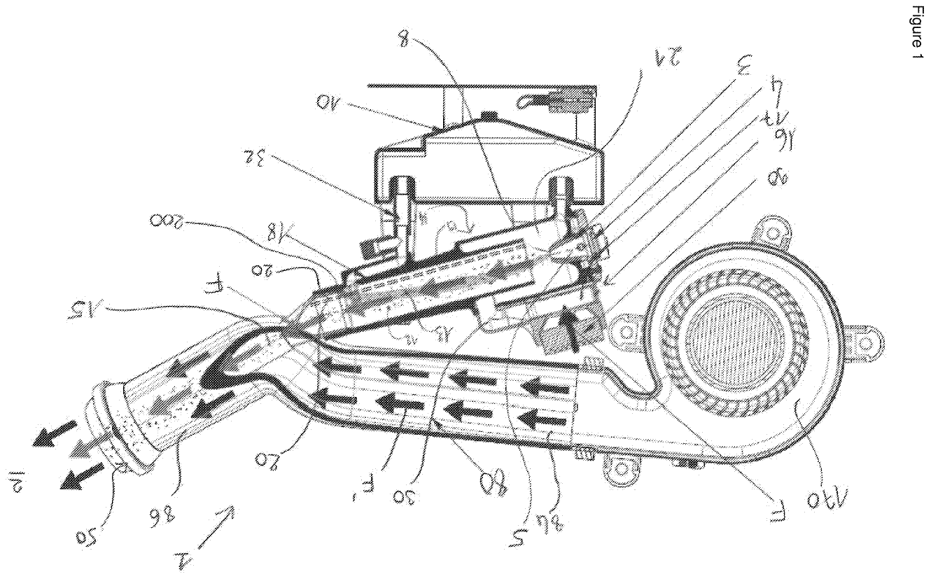

FIG. 1 shows a schematic view in longitudinal section of a nebulizer system according to a first embodiment of the present invention.

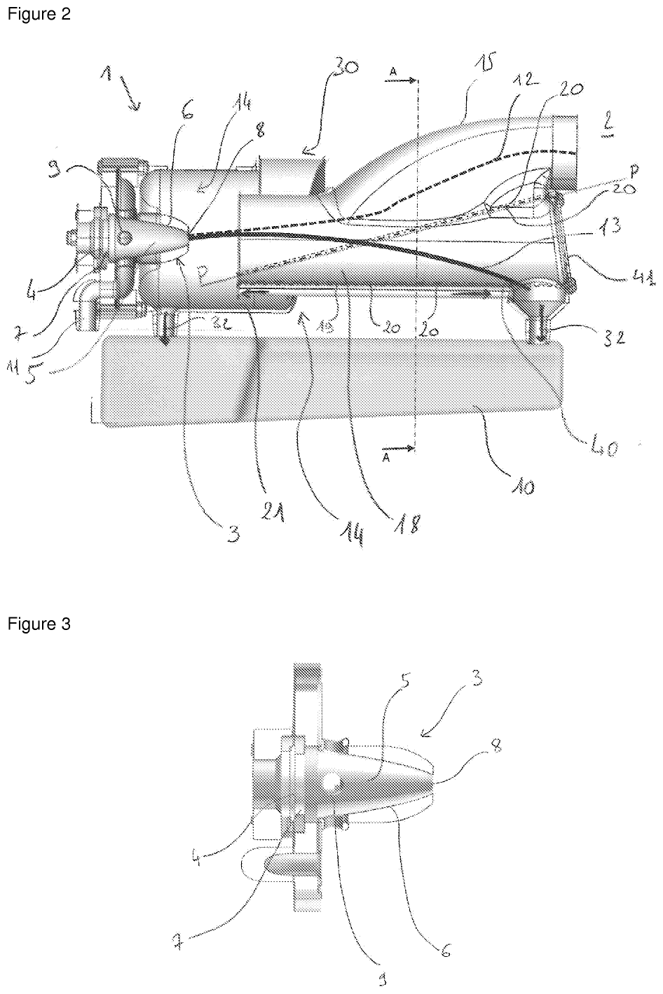

FIG. 2 shows a schematic view in longitudinal section of a nebulizer system according to a second embodiment of the present invention.

FIG. 3 shows an enlarged view of a nebulizer nozzle of the nebulizer system according to FIG. 2.

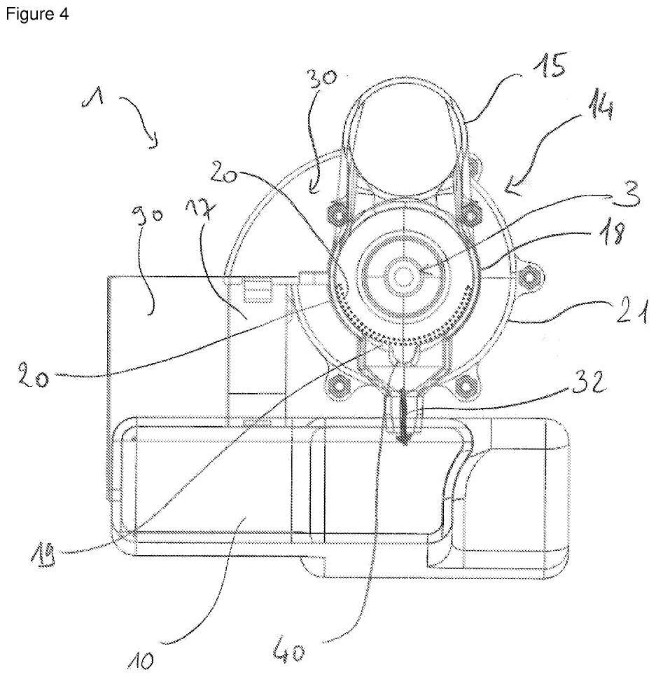

FIG. 4 shows a view in cross section on the axis A-A of a variant of the nebulizer system in FIG. 2, so as to reveal the arrangement of the gratings inside the nebulizer tube.

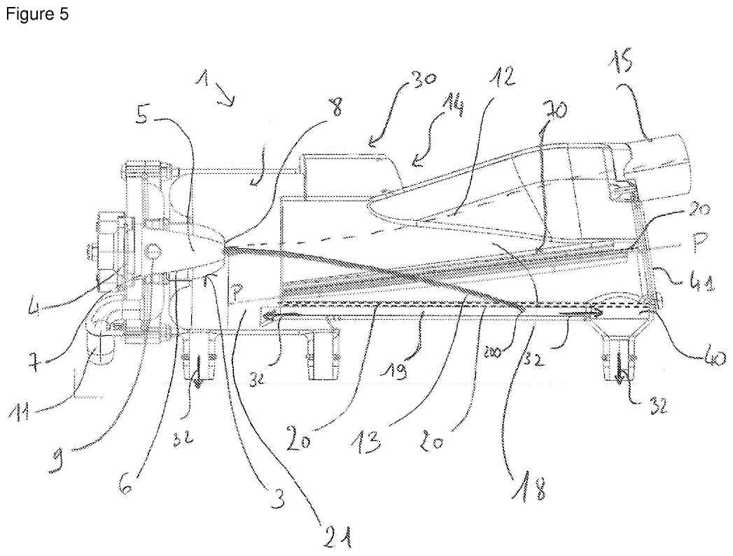

FIG. 5 shows a schematic and simplified view (the liquid reservoir and the hydraulic circuit not being shown), in longitudinal section, of an embodiment variant of the nebulizer system in FIG. 3.

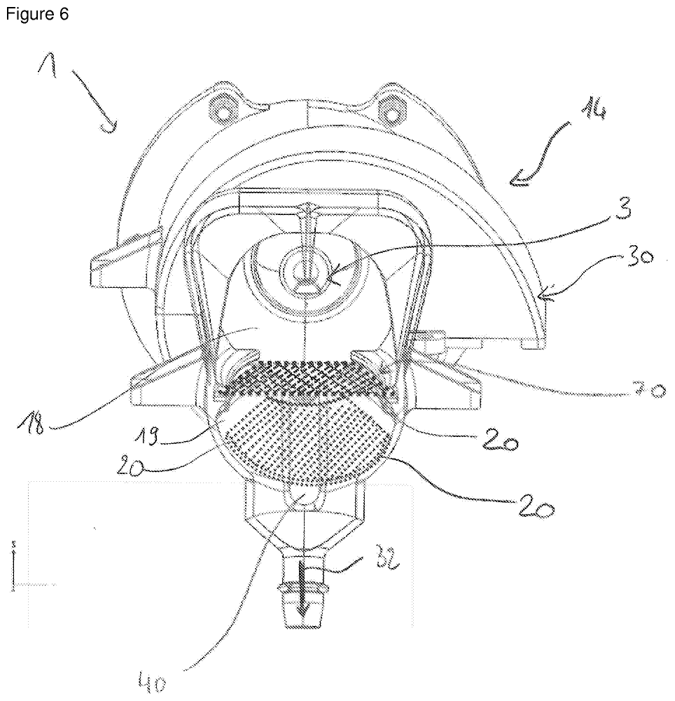

FIG. 6 shows a frontal view in cross section of the nebulizer system according to FIG. 5, so as to reveal the arrangement of the gratings inside the nebulizer tube.

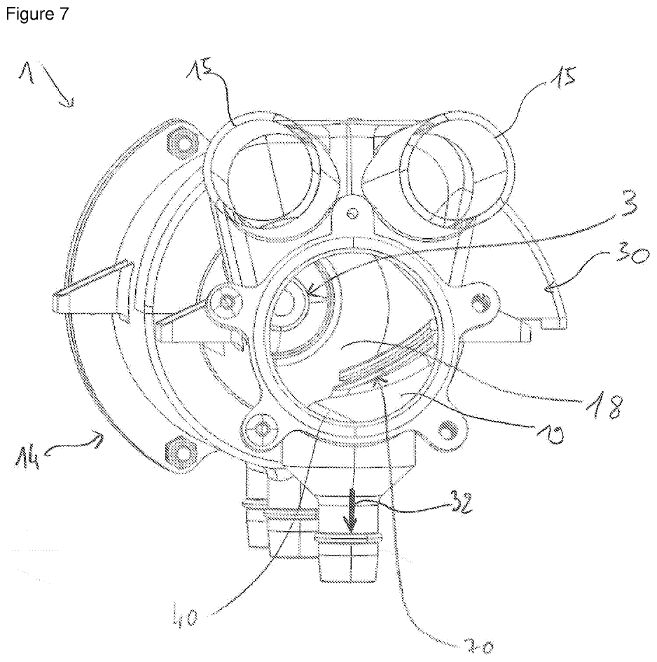

FIG. 7 shows a frontal view in cross section of the nebulizer system according to FIG. 5, so as to reveal the mist driving ducts of such a system.

The invention relates to a nebulizer system 1 for a motor vehicle.

The function of the nebulizer system 1 is to humidify an air flow F′ intended to pass into the interior 2 of a motor vehicle. The nebulizer system 1 makes it possible to cool the vehicle interior 2 and to improve passenger comfort.

As illustrated in the figures, the nebulizer system 1 has a reservoir 10 for a liquid and a nebulizer enclosure 14. The liquid contained in the reservoir 10 is for example water, which may contain a few drops of essential oil. The reservoir 10 acts as a liquid storage reservoir. In this case, it has a parallelepiped overall shape, but its shape does not limit the present invention.

The liquid contained in the reservoir 10 is intended to be nebulized by the nebulizer system 1.

The nebulizer enclosure 14 is realized in this case in the form of an elongate hollow body. It may extend on a plane that is inclined with respect to the longitudinal axis, as illustrated in FIG. 1, or on a plane that is parallel to the longitudinal axis, as can be seen in FIGS. 2 and 5.

The nebulizer enclosure 14 is provided with a nebulizer nozzle 3. The nebulizer nozzle 3 is provided with a device 4 for emitting acoustic waves, for example a piezoelectric element 4. The piezoelectric element may be a quartz crystal.

The device 4 for emitting acoustic waves is configured such that the liquid coming from the reservoir 10 forms a liquid jet 13 and a mist 12 of droplets, the mist 12 being intended to pass into a motor vehicle interior 2.

In the examples illustrated, the nebulizer nozzle 3 also comprises a concentrator for the sound waves generated by the piezoelectric element 4, which is also known as an acoustic concentrator and bears the reference 5 in the following text.

The acoustic concentrator 5 known to those skilled in the art comprises an enclosure 6 that narrows gradually from a rear wall 7 to an outlet orifice 8. The acoustic concentrator 5 also comprises at least one inlet orifice 9 through which the concentrator is fed with liquid coming from the reservoir 10, via a liquid inlet circuit 11 that can be seen in FIGS. 2 and 5.

As can be seen in FIG. 3, the piezoelectric element 4 is disposed against the rear wall 7 of the acoustic concentrator 5.

Thus, the enclosure 6 is fed with liquid via at least one inlet orifice 9 by way of a liquid distribution circuit comprising the reservoir 10 and advantageously a pump (not shown in the figures).

In a known way, the acoustic waves generated by the piezoelectric element 4 are transmitted to the liquid contained in the enclosure 6 through the rear wall 7. The convergent shape of the enclosure 6 makes it possible to focus the acoustic waves in the outlet orifice 8, thereby producing a mist 12 of droplets around the liquid jet 13. This mist 12 is depicted by a set of points in FIG. 1 and by a dashed line in FIGS. 2 and 5. By way of the outlet orifice 8, the mist 12 is directed toward the interior 2 of the motor vehicle via an outlet duct 15 for the mist 12, while a liquid jet 13 flows into the storage reservoir 10 via a liquid recovery circuit 32 depicted by arrows in the figures.

As can be seen in FIG. 1, the nebulizer system 1 also comprises an inlet module 16 for a first air flow F depicted by the arrows F in FIG. 1. The inlet module 16 for the first air flow F has a fan 17 for setting the first air flow in motion, and a filter 90 that is able to filter this first air flow before it enters the nebulizer enclosure 14.

The nebulizer system 1 illustrated in FIGS. 2 to 7 also has an air injection blower housing 30 that is able to channel the air coming from the fan 17 for setting the first air flow in motion in the direction of the interior volume of the nebulizer enclosure 14. The air injection blower housing 30 makes it possible in particular to swirl the first air flow F around the nebulizer enclosure 14 so as to feed said nebulizer enclosure 14 even more effectively.

In the illustrated examples that are representative of the present invention, each nebulizer system 1 comprises acoustic attenuation means 20.

The acoustic attenuation means 20 comprise at least one grating 20.

In the embodiments illustrated in the figures, the nebulizer enclosure 14 comprises a nebulizer chamber 21 and a nebulizer tube 18. The nebulizer nozzle 3 is partially inserted into the nebulizer chamber 21.

The nebulizer tube 18 has an outlet duct 15 for the mist 12.

The acoustic attenuation means 20 will now be described in more detail, first of all in connection with the first embodiment illustrated in FIG. 1.

In the embodiment illustrated in FIG. 1, two gratings 20 are disposed in the nebulizer tube 18 of the nebulizer system 1.

The term “disposed” should be understood as meaning that the gratings 20 are situated in the volume defined by the walls of the nebulizer tube 18.

The two gratings 20 are disposed opposite one another, on mutually parallel planes. The two gratings 20 are, for example, placed in contact with one another. In other words, the two gratings 20 are superposed. The gratings 20 are offset at an angle of 45° with respect to one another in order to attenuate the flow noise even better.

In a variant that is not shown, the two gratings 20 are disposed at a distance from one another.

The two gratings 20 are held in the nebulizer tube 18 for example by adhesive bonding or by way of fixing means, for example spikes formed integrally with the nebulizer tube 18.

The grating 20 is made for example of flexible plastic (insect screen type) or rigid plastic.

The grating 20 may also be made of metal or metal alloy, for example steel.

The grating 20 may be produced from woven threads. In this case, the grating 20 is in the form of a netting.

The grating 20 may also be produced from a perforated metal sheet.

The type of mesh used does not limit the present invention, it being possible for the mesh to be of the circular, triangular, rectangular, square, hexagonal type or any other shape chosen at random.

The gratings 20 used in the nebulizer system 1 as illustrated may be made of the same materials or different materials.

It has been found, in a set of tests, that the two gratings 20 allow the noise generated during the impact of the liquid jet 13 on the walls of the nebulizer system 1 to be made imperceptible, in particular when the liquid jet 13 strikes a bottom wall 19 of the nebulizer tube 18.

The bottom wall 19 is one of the walls of the nebulizer tube 18 that is hit by the liquid jet 13.

It must be understood that the liquid jet 13 is the part of the liquid that is not nebulized at the outlet of the nebulizer chamber 21 and is formed generally of drops that are too large to be entrained by the first air flow F. Drops that are too large will be understood as being drops with a diameter greater than 10 μm.

The gratings 20 make it possible to reduce the speed of the liquid jet 13 at the impact point 200 situated on the bottom wall 19 of the nebulizer tube 18. The user of the nebulizer system 1 is no longer bothered by the noise generated when the jet 13 hits the bottom wall 19.

The liquid that is not nebulized then flows along the bottom wall 19 and is then discharged from the nebulizer enclosure 14 via the liquid recovery circuit 32, toward the storage reservoir 10.

The circuit for the air flow circulating in the nebulizer system 1 according to the invention will now be described.

An air flow enters the nebulizer system 1 by way of the inlet module 16 for the first air flow F and, in particular, through the filter 90 for the first air flow F, then passes through the fan 17 for setting the first air flow F in motion.

Subsequently, the air flow F arrives in the air injection blower housing 30 before passing into the nebulizer enclosure 14 at the nebulizer chamber 21. The air injection blower housing 30 makes it possible to introduce the first air flow F into the nebulizer chamber 21 perpendicularly to the longitudinal axis of the nebulizer enclosure 14.

The first air flow F introduced into the nebulizer chamber 21 is then directed parallel to the longitudinal axis of the nebulizer enclosure 14 and in counter-current to the direction taken by the liquid nebulized by the nebulizer nozzle 3, specifically until it comes into contact with redirection means (not shown). The redirection means then orient the air substantially parallel to the direction taken by the liquid nebulized in the nebulizer nozzle 3.

The mixture of the first air flow F and the nebulized liquid is produced in the nebulizer enclosure 14, in particular in the nebulizer chamber 21. Thus, the first air flow F helps to expel the mist 12 of droplets from the nebulizer system 1.

As indicated above, the nebulizer system 1 also has an outlet duct 15 for the mist.

In the example illustrated in FIG. 1, the outlet duct 15 for the mist is disposed in the continuation of the nebulizer tube 18 and is slightly inclined with respect to the axis of extension of the nebulizer tube 18. The outlet duct 15 for the mist extends in this case as far as a terminal orifice fluidically connected to at least one air vent 50 of the motor vehicle and, thus, the interior 2 of this same vehicle.

The particular embodiment illustrated in FIG. 1 provides for the nebulizer system 1 to have an intake system 80 for a second air flow F′.

The intake system 80 for the second air flow F′ is made up of an air inlet (not shown), of a fan 170 for setting the second air flow F′ in motion, of a duct 84 for carrying the second air flow toward the air vent 50, and of an outlet 86.

The second air flow F′ could be drawn in, for example, via a spur in the interior 2 of the motor vehicle.

The outlet 86 of the intake system 80 for the second air flow F′ is disposed, in the embodiment illustrated in FIG. 1, in the region of the air vent 50. The air vent 50 defines a mixing zone for the mist 12 of droplets coming from the outlet duct for the mist 34 and for the second air flow F′ coming from the intake system 80.

The acoustic attenuation means 20 will now be described in more detail in connection with the second embodiment illustrated in FIGS. 2 and 5.

Just like in the example in FIG. 1, the acoustic attenuation means 20 comprise two gratings 20 disposed on the bottom wall 19 of the nebulizer tube 18.

The bottom wall 19 of the nebulizer tube 18 also comprises a channel 40 intended to receive the liquid jet 13 output by the nebulizer nozzle 3.

The channel 40 is particularly visible in FIGS. 4 and 6.

The two gratings 20 at least partially conform to the shape of the channel 40.

The number of layers of gratings 20 can be altered and does not limit the present invention.

Advantageously, the nebulizer system 1 also has at least one grating 20 extending in a plane 23 that is inclined with respect to the bottom wall 19 of the nebulizer tube 18.

This configuration is possible since, in the nebulizer system 1 in relation to the second embodiment of the invention, the outlet duct 15 for the mist is not situated in the continuation of the nebulizer tube 18 but diverges from the nebulizer enclosure 14. Thus, it is possible to add gratings 20 across the nebulizer tube 18 without these stopping the mist 12 of droplets generated. An increase in acoustic attenuation is thus observed as a result of a further reduction in the speed of the liquid jet 13. Moreover, by virtue of this relative disposition of the nebulizer tube 18 and the outlet duct 15 for the mist, the system is more compact as a whole.

As can be seen in FIG. 4, the nebulizer tube 18 has a circular cross section. It is closed, at its end farthest away from the nebulizer nozzle 3, by a cover 41. The cover 41 makes it possible to avoid losses of the liquid that is not nebulized. It can be fixed to the nebulizer tube 18 for example with the aid of screws. In different cross-sectional views of the system, the cover has not been shown in order to reveal the gratings inside the nebulizer tube 18.

In a variant illustrated in FIGS. 5 to 7, the nebulizer tube 18 also comprises a guide means 70 protruding from one of the walls of said tube. In the example illustrated, this guide means is a slideway 70 formed integrally with the wall of the nebulizer tube 18. In a nonlimiting way, other guide means can be envisioned, for example a set of bearing points arranged on one of the walls of the nebulizer tube.

The slideway 70 is intended to guide the grating 20 along the inclined plane 23.

The set of three gratings 20 disposed in the nebulizer system 1 illustrated in FIGS. 5 to 7 has the function of braking the liquid jet 13 coming from the nebulizer nozzle 3.

As illustrated in FIG. 7, the nebulizer system 1 has two outlet ducts 15 for the mist.

By virtue of the present invention, it is possible to eliminate the noise caused by the liquid jet, and in particular when the non-nebulized liquid jet strikes the bottom wall of the nebulizer tube. In this way, the comfort of the users is significantly improved.

The nebulizer system 1 can be integrated into a motor vehicle, for example into a central console. In particular, the nebulizer system 1 could be disposed in a housing situated above and/or between ventilation ducts at the back of and below the front seat armrests.

Provision may be made for this nebulizer system 1 to be intended for the front-seat passengers of the motor vehicle and thus to be disposed at the front of the latter.

1. A nebulizer system for a motor vehicle, said system having:
at least one reservoir for a liquid;
at least one nebulizer enclosure provided with a nebulizer nozzle, said nozzle being provided with a device for emitting acoustic waves,
said nebulizer enclosure being configured such that the liquid coming from the reservoir forms a liquid jet and a mist of droplets, said mist being configured intended to pass into a motor vehicle interior; and
at least one acoustic attenuation means comprising at least one grating.

2. The nebulizer system as claimed in claim 1, wherein said nebulizer enclosure comprises a nebulizer chamber and a nebulizer tube, said nebulizer nozzle being inserted at least partially into said nebulizer chamber and said at least one grating being disposed at least partially in the nebulizer tube.

3. The nebulizer system as claimed in claim 2, wherein said at least one grating conforms to the shape of at least one of the walls of the nebulizer tube.

4. The nebulizer system as claimed in claim 3, wherein said wall is a bottom wall of the nebulizer tube.

5. The nebulizer system as claimed in claim 4, wherein said bottom wall of the nebulizer tube comprises a channel configured to receive the liquid jet output by the nebulizer nozzle.

6. The nebulizer system as claimed in claim 5, wherein the grating at least partially conforms to the shape of the channel.

7. The nebulizer system as claimed in claim 4, wherein said at least one grating extends in a plane that is inclined with respect to said bottom wall of the nebulizer tube.

8. The nebulizer system as claimed in claim 2, wherein the nebulizer tube comprises at least one guide means protruding from one of its walls, said guide means being intended to guide the grating along the inclined plane.

9. The nebulizer system as claimed in claim 8, wherein said guide means is a slideway.

10. A ventilation, heating and/or air-conditioning device for a motor vehicle, comprising a nebulizer system as claimed in claim 1.
